SEQUENCE LISTING (13 sequences)

SEQ ID NO: 1 - protein, 127 aa, Homo sapiens
  HEPV
IKGDRGEIGPPGPRGEDGPEGPKGRGGPNGDPGPLGPPGEKGKLGVPGLPGYPGRQGPKGSIGFPGFPGANGEKGGRGTPGKPGPRGQRGPTGPRGERGPRGITGKPGPKGNSGGDGPAGPPGERGP

SEQ ID NO: 2 - protein, 20 aa, Artificial Sequence
  MISC_FEATURE (1),(3)..(5), (7)..(8) and (10)..(20): Synthetic peptide in which Xaa at positions 1, 3-5, 7, 8 and 10-20 may be any amino acid
XKXXXRXXRXXXXXXXXXXX

SEQ ID NO: 3 - protein, 20 aa, Homo sapiens
GKPGPRGQRGPTGPRGERGP

SEQ ID NO: 4 - protein, 127 aa, Artificial Sequence
  Synthetic Mutant of HEPV
IKGDRGEIGPPGPRGEDGPEGPKGRGGPNGDPGPLGPPGEKGKLGVPGLPGYPGRQGPKGSIGFPGFPGANGEKGGRGTPGAPGPAGQAGPTGPRGERGPRGITGKPGPKGNSGGDGPAGPPGERGP

SEQ ID NO: 5 - protein, 1838 aa, Homo sapiens
MDVHTRWKARSALRPGAPLLPPLLLLLLWAPPPSRAAQPADLLKVLDFHNLPDGITKTTGFCATRRSSKGPDVAYRVTKDAQLSAPTKQLYPASAFPEDFSILTTVKAKKGSQAFLVSIYNEQGIQQIGLELGRSPVFLYEDHTGKPGPEDYPLFRGINLSDGKWHRIALSVHKKNVTLILDCKKKTTKFLDRSDHPMIDINGIIVFGTRILDEEVFEGDIQQLLFVSDHRAAYDYCEHYSPDCDTAVPDTPQSQDPNPDEYYTEGDGEGETYYYEYPYYEDPEDLGKEPTPSKKPVEAAKETTEVPEELTPTPTEAAPMPETSEGAGKEEDVGIGDYDYVPSEDYYTPSPYDDLTYGEGEENPDQPTDPGAGAEIPTSTADTSNSSNPAPPPGEGADDLEGEFTEETIRNLDENYYDPYYDPTSSPSEIGPGMPANQDTIYEGIGGPRGEKGQKGEPAIIEPGMLIEGPPGPEGPAGLPGPPGTMGPTGQVGDPGERGPPGRPGLPGADGLPGPPGTMLMLPFRFGGGGDAGSKGPMVSAQESQAQAILQQARLALRGPAGPMGLTGRPGPVGPPGSGGLKGEPGDVGPQGPRGVQGPPGPAGKPGRRGRAGSDGARGMPGQTGPKGDRGFDGLAGLPGEKGHRGDPGPSGPPGPPGDDGERGDDGEVGPRGLPGEPGPRGLLGPKGPPGPPGPPGVTGMDGQPGPKGNVGPQGEPGPPGQQGNPGAQGLPGPQGAIGPPGEKGPLGKPGLPGMPGADGPPGHPGKEGPPGEKGGQGPPGPQGPIGYPGPRGVKGADGIRGLKGTKGEKGEDGFPGFKGDMGIKGDRGEIGPPGPRGEDGPEGPKGRGGPNGDPGPLGPPGEKGKLGVPGLPGYPGRQGPKGSIGFPGFPGANGEKGGRGTPGKPGPRGQRGPTGPRGERGPRGITGKPGPKGNSGGDGPAGPPGERGPNGPQGPTGFPGPKGPPGPPGKDGLPGHPGQRGETGFQGKTGPPGPPGVVGPQGPTGETGPMGERGHPGPPGPPGEQGLPGLAGKEGTKGDPGPAGLPGKDGPPGLRGFPGDRGLPGPVGALGLKGNEGPPGPPGPAGSPGERGPAGAAGPIGIPGRPGPQGPPGPAGEKGAPGEKGPQGPAGRDGLQGPVGLPGPAGPVGPPGEDGDKGEIGEPGQKGSKGDKGEQGPPGPTGPQGPIGQPGPSGADGEPGPRGQQGLFGQKGDEGPRGFPGPPGPVGLQGLPGPPGEKGETGDVGQMGPPGPPGPRGPSGAPGADGPQGPPGGIGNPGAVGEKGEPGEAGEPGLPGEGGPPGPKGERGEKGESGPSGAAGPPGPKGPPGDDGPKGSPGPVGFPGDPGPPGEPGPAGQDGPPGDKGDDGEPGQTGSPGPTGEPGPSGPPGKRGPPGPAGPEGRQGEKGAKGEAGLEGPPGKTGPIGPQGAPGKPGPDGLRGIPGPVGEQGLPGSPGPDGPPGPMGPPGLPGLKGDSGPKGEKGHPGLIGLIGPPGEQGEKGDRGLPGPQGSSGPKGEQGITGPSGPIGPPGPPGLPGPPGPKGAKGSSGPTGPKGEAGHPGPPGPPGPPGEVIQPLPIQASRTRRNIDASQLLDDGNGENYVDYADGMEEIFGSLNSLKLEIEQMKRPLGTQQNPARTCKDLQLCHPDFPDGEYWVDPNQGCSRDSFKVYCNFTAGGSTCVFPDKKSEGARITSWPKENPGSWFSEFKRGKLLSYVDAEGNPVGVVQMTFLRLLSASAHQNVTYHCYQSVAWQDAATGSYDKALRFLGSNDEEMSYDNNPYIRALVDGCATKKGYQKTVLEIDTPKVEQVPIVDIMFNDFGEASQKFGFEVGPACFMG

SEQ ID NO: 6 - DNA, 35 bp, Artificial Sequence
  Synthetic oligonucleotide
gcgcccaggaccggcgggggcaggcaggcccaacg

SEQ ID NO: 7 - DNA, 33 bp, Artificial Sequence
  Synthetic Oligonucleotide
cgttgggcctgcctgccccgccggtcctggcgc

SEQ ID NO: 8 - DNA, 20 bp, Artificial Sequence
  Synthetic Primer
ctggtccaagaggatttcca

SEQ ID NO: 9 - DNA, 20 bp, Artificial Sequence
  Synthetic Primer
tcattgccttgcacgtagag

SEQ ID NO: 10 - DNA, 18 bp, Artificial Sequence
  Synthetic Primer
gcgccaaaggagaagtgg

SEQ ID NO: 11 - DNA, 21 bp, Artificial Sequence
  Synthetic Primer
tttcagcctccaactgaagaa

SEQ ID NO: 12 - DNA, 19 bp, Artificial Sequence
  Synthetic Primer
atggggaaggtgaaggtcg

SEQ ID NO: 13 - DNA, 20 bp, Artificial Sequence
  Synthetic Primer
taaaagcagccctggtgacc

(19) United States
(12) Patent Application Publication
(10) Pub. No.: US 2017/0327561 A1
(43) Pub. Date: Nov. 16, 2017

(54) Anti-Angiogenic Properties of Collagen V Derived Fragments

(71) Applicants: Ecole Normale Superieure de Lyon, cedex 07 Lyon (FR); Institut National de la Sante et de la Recherche Medicale (INSERM), Paris (FR); Universite Claude Bernard Lyon I, Villeurbanne Cedex (FR); Centre National de la Recherche Scientifique, Paris Cedex 16 (FR); Universite Grenoble Alpes, Saint Martin d'Heres (FR)

(72) Inventors: Florence Ruggiero, Villeurbanne (FR); Rachel Manuel, Randwick, New South Wales (AU); Jean-Luc Coll, La Tronche (FR); Michelle Keramidas, Meylan (FR)

(21) Appl. No.: 15/531,143

(22) PCT Filed: Dec. 2, 2014
(86) PCT No.: PCT/EP2014/076185

§ 371 Date: May 26, 2017

(51) Int. Cl. C07K 14/78; A61K 49/00; A61K 38/39; G01N 33/574; A61K 45/06

(52) U.S. Cl. CPC C07K 14/78; G01N 33/574; A61K 45/06; A61K 38/39; A61K 49/0056; G01N 2333/503; G01N 2333/78

(57) ABSTRACT

The present invention relates to a peptide comprising an amino acid sequence at least 85% identical to the amino acid sequence as shown in SEQ ID NO. 1, wherein the residues Lys⁹⁰⁵, Arg⁹⁰⁹, and Arg⁹¹² contained therein are present, for use as a medicament, in particular for its use as an inhibitor of FGF-2 induced angiogenesis.

The present invention relates to the field of angiogenesis process, specifically the FGF-2 induced angiogenesis. The present invention relates more particularly to the inhibition of the angiogenesis process in the field of cancer therapy.

### PRIOR ART

Angiogenesis, the process of new blood-vessel growth, has an essential role in development, reproduction and repair. However, pathological angiogenesis occurs not only in tumor formation, but also in a range of non-neoplastic diseases that could be classed together as ‘angiogenesis-dependent diseases’.

Angiogenesis plays a pivotal role in tumor growth and metastasis. Indeed, angiogenic factors are overexpressed in tumors. Significant efforts have been undertaken to develop anti-angiogenic strategies for cancer therapy.

The patent application US 2014/0100164 describes peptides presenting anti-angiogenic activities. General peptides motifs associated with anti-angiogenic activity were identified from three families of human proteins: type I thrombospondin domain containing proteins, CXC chemokines and collagens. A peptide issued from the collagen type IV was identified as presenting anti-angiogenic activity.

The patent application US 2013/0316950 also describes peptides derived from collagen IV and their use for limiting angiogenesis in cancers.

The vascular endothelial growth factor (VEGF) plays a central role in the angiogenesis phenomena. Therefore, agents that selectively target VEGF and its receptors have been investigated, and have shown promising activity in clinical trials. In particular, anti-angiogenesis drugs have been developed under the names Avastin® and Endostar®.

However, in both preclinical and clinical settings, the benefits of these treatments are at best transitory, and are followed by a restoration of tumor growth and progression. Indeed it appears that some patients ultimately develop resistance to these drugs. One proposed mechanism for this resistance is the up-regulation in tumoral tissues of other pro-angiogenic factors, in particular of the fibroblast-growth factor 2 (FGF-2).

FGF-2, also known as βFGF, FGF2, FGF-β or basic fibroblast growth factor, belongs to the family of the heparin-binding fibroblast-growth factors. FGF-2 interacts with endothelial cells through two distinct classes of receptors, the high affinity tyrosine-kinase receptors (FGFRs) and low affinity heparan sulfate proteoglycans (HSPGs), present on the cell surface and in the extracellular matrix. FGF-2 acts on endothelial cells, during wound healing of normal tissues, and during tumor development. When the VEGF pathway is blocked by an anti-angiogenic drug, an FGF-2 up-regulation is observed, allowing tumor vascularization and re-growth.

To prevent this “tumor evasion” from anti-VEGF therapy, research has focused on the development of new anti-angiogenesis methods and drugs, in particular directed against FGF-2-mediated angiogenesis.

FGF-2 antagonist long-pentraxin 3 (PTX3) has been shown to bind FGF-2 with high affinity and specificity. Synthetic peptides derived from PTX3, targeting directly FGF-2, show an anti-angiogenic activity (Alessi et al., 2009).

Efforts have been made in order to identify other synthetic peptides showing significant and specific anti-FGF-2-mediated-angiogenesis activity.

### SUMMARY OF THE INVENTION

Surprisingly, inventors have now identified a peptide derived from the human collagen V proα1 chain, that can be used as a medicament.

This peptide may be used as a medicament, in particular as an inhibitor of FGF-2-induced biological effects, and more particularly as an inhibitor of FGF-2 induced angiogenesis process, notably for treating cancer. Indeed, this peptide presents specific anti-angiogenic properties, when administered to animals or patients.

The peptide is characterized as comprising an amino acid sequence at least 85% identical to the amino acid sequence as shown in SEQ ID NO. 1, wherein the residues Lys⁹⁰⁵, Arg⁹⁰⁹, and Arg⁹¹² contained therein are present.

This peptide is derived from the fragment [Ile⁸²⁴ to Pro⁹⁵⁰] of α1 chain from collagen V, and for more clarity the numbering of amino acids in the complete chain α1(V) (the pro-α1(V) chain) has been conserved.

In a specific embodiment, the peptide is the peptide ‘HEPV’, a 12 kDa fragment of the collagen V pro-α1 chain consisting in the residues Ile⁸²⁴ to Pro⁹⁵⁰, that has been previously described as a peptide binding to heparin (Delacoux et al., 1998; Delacoux et al., 2000; Ricard-Blum et al., 2006).

A pharmaceutical composition and a kit-of-parts, comprising this peptide, are also objects of the present application.

A peptide comprising an amino acid sequence at least 85% identical to the amino acid sequence as shown in SEQ ID NO. 1 [Ile⁸²⁴-Pro⁹⁵⁰], wherein the residues Lys⁹⁰⁵, Arg⁹⁰⁹, and Arg⁹¹² contained therein are present, coupled with a detectable label is also an object of the invention.

The present application also relates to a method for imaging angiogenesis sites of an animal or a human individual, comprising the step of detecting the label of a peptide as defined above, that has been previously administered to the said animal or to the said human individual.

### BRIEF DESCRIPTION OF THE DRAWINGS

FIG. 1. Structure of peptide HEPV derived from the chain proα1(V) A. Structure of the procollagen V heterotrimer [α1(V)]₂α2(V). CRR, cysteine-rich repeats domain; VR, variable region; TSPN, thrombospondin N-terminal like domain.

B. Structure of the proα1(V) and proα2(V) chains. Black bars represent non-collagenous domain (NC2) located between the small triple helix domain and the major triple helix domain.

C. Amino-acid sequence of the fragment HEPV. The basic residues arginine and lysine are in bold. The sequence responsible for heparin binding is underlined.

FIG. 2. HEPV stimulates the expression of collagens IV and XVIII al chains

Expression of COL14A1 and COL18A1 mRNA in human dermal microvascular endothelial cells (HDMEC) treated with HEPV for 4, 12 and 24 hours, analyzed by real-time PCR. Values are normalized to the house keeping gene L30. Quantification is expressed relative to controls (cells cultivated in the same conditions without HEPV). Values are mean±SEM (n=3).

FIG. 3. Production of a non-functional mutant derived from the peptide HEPV, HEPV-AHBS A. SDS PAGE analysis of HEPV-AHBS. Lane 1, E. coli bacterial lysate containing recombinant HEPV-AHBS. Lane 2, HEPV-ΔHBS-containing fraction after cation exchange chromatography. Lane 3, purified HEPV-ΔHBS containing fraction after the second step of purification using cation exchange chromatography.

B. Affinity of HEPV and HEPV-ΔHBS for heparin. The HEPV basic residues which have been mutated in alanine are underlined. Only the region G⁹⁰¹-P⁹²³ of the fragment that contains the heparin binding site is shown. Purified HEPV and HEPV-ΔHBS were passed through a heparin-sepharose affinity chromatography and eluted with a NaCl gradient (dotted line). HEPV is eluted with 0.35 M NaCl while HEPV-ΔHBS is eluted with 0.2 M NaCl.

FIG. 4. HEPV inhibits FGF-2-induced ERK1/2 and Akt phosphorylation in endothelial cells. Western blots of endothelial cell lysates treated with FGF-2 (A) or VEGF (B) in presence of HEPV or HEPV-ΔHBS with antibodies to ERK1/2, p-ERK1/2, Akt and p-Akt, and quantifications. The phosphorylated p-ERK1/2 and p-Akt proteins are detected with specific antibodies.

FIG. 5. HEPV acts on formation of blood vessels in mouse

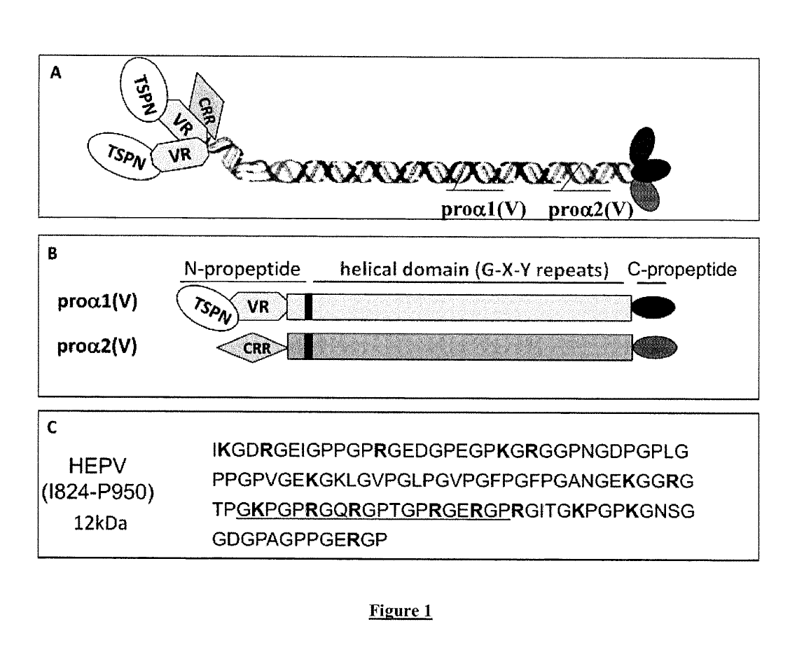

(A) Ability of the fluorescent peptides HEPV and HEPV-ΔHBS to recognize angiogenesis sites. Sponges impregnated with FGF-2 or PBS are implanted in nude mice. After intravenous injection of the Alexa700 labeled fluorescent peptides, their accumulation in angiogenic areas of the sponges is quantified using 2D fluorescence reflectance imaging (see arrows). The relative intensities of emitted fluorescent light in the sponges are then calculated and expressed as Reference Light Units (RLU) during 200 ms.

(B) Formation of new blood vessels in nude mice implanted with a sponge impregnated with FGF-2 or PBS. After repeated treatments with peptides HEPV or HEPV-ΔHBS, the sponges are extracted and the presence of hemoglobin quantified. The presence of hemoglobin reflects the blood vessels content, as documented by the photos.

FIG. 6. HEPV affects the tumor growth of implanted tumors in nude mice.

(A) Murine Tsa/Pc breast cancer cells implanted subcutaneously are treated from day 5 repeatedly by peritumoral injections of 50 μl PBS containing 50 μg control (HEPV-ΔHBS) of HEPV peptides every 2 days. As can be observed, tumor growth is significantly (p<0.05) slowed down in HEPV treated animals. Tumors sections were immunostained using an anti-CD31 antibody that detects blood vessels (B) or an anti-Ki67 antibody that stains proliferating cells (C).

Formation of new blood vessels is inhibited in breast tumors implanted in nude mice during treatment with peptides HEPV, especially during the first 20 days. As well, this treatment reduces the number of proliferating tumor cells.

### DETAILED SPECIFICATION OF THE INVENTION

All technical terms used in the present specification are well known by the man skilled in the art, and are extensively defined in the reference manual from Sambrook et al. entitled <<Molecular Cloning: a Laboratory Manual>>.

The present invention is related to a peptide comprising an amino acid sequence at least 85% identical to the amino acid sequence as shown in SEQ ID NO. 1, wherein the residues Lys⁹⁰⁵, Arg⁹⁰⁹, and Arg⁹¹² contained therein are present, for use as a medicament.

The residues are numerated according to their position in the complete sequence of the proα1(V) chain of the Collagen V, comprising 1838 residues, as shown in SEQ ID NO. 5.

The sequence SEQ ID NO. 1 represents the sequence of a peptide derived from the human collagen proα1(V) chain, comprising 127 residues starting with an isoleucine at the position 824, and finishing with a proline at the position 950, as underlined in SEQ ID 5.

The phrase “an amino acid sequence at least 85% identical to the amino acid sequence as shown in SEQ ID NO. 1” designates a candidate sequence sharing 85% amino acid identity with the reference sequence. This requires that, following alignment, 85% of the amino acids in the candidate sequence are identical to the corresponding amino acids in the reference sequence.

By ‘identity of amino acid’ is meant that the same amino acid is observed on both sequences. Identity does not take account of post-translation modifications that may occur on amino acids; for example, an hydroxylated proline is considered as being identical to a non-hydroxylated proline.

Identity according to the present invention is determined by aid of computer analysis, such as the ClustalW computer alignment program, and the default parameters suggested therein. The ClustalW software is available from the website http://www.clustal.org/clustal2/. By using this program with its default settings, the part of a query and of a reference polypeptide are aligned. The number of fully conserved residues are counted and divided by the length of the reference polypeptide.

The terms “at least 85%” indicates that the percentage of identity between both sequences, the query and the reference polypeptide of sequence SEQ ID NO. 1, is of at least 85, 86, 87, 88, 89, 90, 91, 92, 93, 94, 95, 96, 97, 98, 99 or 100%.

In particular, the amino acid sequence at least 85% identical to the amino acid sequence as shown in SEQ ID NO. 1 presents at least 90% of identity with SEQ ID NO. 1.

In particular, the amino acid sequence at least 85% identical to the amino acid sequence as shown in SEQ ID NO. 1 presents at least 95% of identity with SEQ ID NO. 1.

In particular, the amino acid sequence at least 85% identical to the amino acid sequence as shown in SEQ ID NO. 1 presents at least 98% of identity with SEQ ID NO. 1.

According to the invention, the amino acid sequence showing at least 85% of identity with the amino acid sequence as shown in SEQ ID NO. 1, presents the following conserved residues: Lys⁹⁰⁵, Arg⁹⁰⁹, and Arg⁹¹². These residues are essential for the specificity and activity of the peptide, and cannot be modified under the risk to change the specificity and/or activity of the peptide.

In an embodiment of the invention, the amino acid sequence showing at least 85% of identity with the amino acid sequence as shown in SEQ ID NO. 1, presents the following conserved residues: Lys⁹⁰⁵, Arg⁹⁰⁹, Arg⁹¹², Arg⁹¹⁸ and Arg⁹²¹. The presence of these amino acids, involved in the heparin binding site, might also be important for the activity of the peptide as a medicament.

Without wishing to be bound by the theory, inventors have observed that the peptide according to the invention binds specifically to heparin and heparan sulfate, both molecules being involved in cell-matrix interactions (Delacoux et al., 2000; Ricard-Blum et al., 2006). If the binding site disappears or is not functional anymore, the FGF-2 signalization pathway is inhibited, as presented in the examples section.

The phrase “for use as a medicament” designates the use of said peptide in therapy, in particular in human therapy.

A “medicament” is synonymous of “pharmaceutical drug”, “medicine”, “medication” or “medicinal product”, and designates an active compound, intended for internal or external use, for curing, treating, or preventing a disease.

Surprisingly, inventors have identified the peptide as described previously, as an active compound that can be used as a medicament. Advantageously, this peptide is non-toxic for animals or humans, since it does not accumulate into the liver after injection into the blood system.

Uses of the Peptide

According to a first embodiment, the peptide according to the invention is used as an inhibitor of FGF-2 induced biological effects on target cells. This embodiment can be performed in vivo or in vitro.

The term “inhibitor” designates the mode of action of the peptide, that reduces or even suppresses the biological activity of the FGF-2 on its target cells. In particular, the biological effects of FGF-2 generally observed are reduced of at least 50%, at least 60%, at least 70%, at least 80%, at least 90%, and in a preferred embodiment the biological effects of FGF-2 on target cells are inhibited at 100%, i.e. they are completely suppressed.

The phrase “FGF-2 induced biological effects on target cells” means all biological effects that are specifically induced by the presence of a sufficient amount of FGF-2. Main effects are formation of new blood vessels, but FGF-2 acts also in the regulation of bone mineralization.

Target cells of FGF-2 are cells expressing receptors able to bind the factor FGF-2, and to transmit the signal to the cells. Two classes of receptors have been identified up to now, the high affinity tyrosine-kinase receptors (FGFRs) and the low affinity heparan sulfate proteoglycans (HSPGs). Target cells are mainly endothelial cells, but also cardiomyocytes and osteoblasts related-cells.

FGF-2 is involved in numerous physiological functions, and therefore a peptide acting as an inhibitor of FGF-2 induced biological effects could be used in the treatment of several diseases, and in particular in the treatment of: glioblastoma multiforme, heart failure, Alzheimer's disease, glomerulosclerosis, and myelofibrosis with myeloid metaplasia.

Glioblastoma multiforme (GBM) is the most malignant form of central nervous system tumor, and current therapies are largely ineffective at treating the cancer. It is also one of the most highly vascularized cancers. Secretion of FGF-2 by GBM cells enhances the blood brain barrier function of endothelial cells, which also contributes to drug resistance in GBM. It is speculated that the presence of glioblastoma stem or stem-like cells (GSCs), a rare type of pluripotent cancer cell that possesses the ability to self-renew and generate tumors, could be an important factor contributing to the resistance to treatment and deadliness of the cancer. It has been shown that FGF-2 plays a significant part in regulating GBM and GSC (Haley and Kim, Cancer letters, 2014)

Heart failures represent a major cause of morbidity and mortality. FGF-2 promotes cardiac hypertrophy and fibrosis by activating MAPK signaling through the activation of FGF receptor 1c (FGFR). Regulating FGF-2 signaling may represent potential therapeutic strategies for heart failure (Itho and Ohta, Front Physiol, 2013)

Neurogenesis persists in the aged human dentate gyrus but its role and regulation in pathological conditions such as Alzheimer's disease (AD), where the neurotrophic environment is changed, are poorly understood. In hippocampal progenitor cells from adult rats, FGF-2 decreased, in a dose-dependent manner, microtubule-associated protein 2, and increased tau levels, indicating an FGF-2-induced dendrite to axon polarity shift. AD pathogenesis might involve an abnormally elevated FGF-2-associated dysregulation of dentate gyrus neurogenesis, especially neuronal polarity. Cerebrolysin, a neurotrophic drug which has been shown to improve cognition and mood of AD patients, was found to increase neuron-like differentiated adult rat hippocampal progenitors in culture both by reducing apoptosis and by counteracting the FGF-2-induced polarity shift (Tatebayashi et al, Acta Neuropathol, 2003). Counteracting FGF-2 activity may represent a promising therapeutic target for this disease.

In kidney, FGF-2 increases glomerular protein permeability and accelerates glomerulosclerosis (Chen et al, Current Vascular Pharmacology, 2004). In glomeruli and neointimae of allografts, a massive accumulation of FGF-2 was observed. Profiling the heparan sulfate polysaccharide side chains revealed conversion from a non-FGF-2-binding heparan sulfate phenotype in control and isografted kidneys toward a FGF2-binding phenotype in allografts. FGF2-induced proliferation is dependent on sulfation and can be inhibited by exogenously added heparan sulfate. Counteracting FGF-2 signaling through the heparin binding fragment HEPV could retard development of glomerulosclerosis and neointima formation in chronic transplant dysfunction (Katta et al, Am J Pathol, 2013).

Myelofibrosis with myeloid metaplasia (MMM) is a myeloproliferative disorder characterized by clonal expansion of hematopoiesis and marrow fibrosis. Previous results have shown an increased production of two potent fibrogenic factors also involved in the regulation of primitive hematopoietic cells, namely transforming growth factor-beta1 (TGF-beta1) and basic fibroblast growth factor (bFGF or FGF-2), in patients with MMM. The myeloproliferation characteristic of this disease may result from an abnormal proliferation of CD34+ hematopoietic progenitors. The very low expression of FGF-2 and its type I and II receptors detected in normal CD34+ cells contrasts with that observed in patients' CD34+ cells, which is significantly higher. The increased expression of FGF-2 and its receptors associated with the reduction of the TGF-beta binding receptor in CD34+ progenitors from MMM patients might facilitate the expansion of hematopoietic progenitors, not only by stimulating their growth and/or survival, but also by overcoming negative regulatory signals (Le Bousse-Kerdiles, Blood, 1996; Le Bousse-Kerdiles and Martyré, Ann Hamatol, 1999). Counteracting FGF-2 activity may represent a promising therapeutic target for this disease.

Other diseases can be treated with the peptide according to the invention, such as the diabetic retinopathy and rheumatoid arthritis. This anti-angiogenic peptide may also be used in the treatment of ocular proliferative diseases, such as age-related macular degeneration.

According to a second embodiment, the peptide according to the invention is used as an inhibitor of FGF-2 induced angiogenesis.

“Angiogenesis” refers to the dynamic process that includes blood vessel formation, blood vessel remodeling, blood vessel stabilization, blood vessel maturation, and establishment of a functional blood vessel network. This process of angiogenesis is induced with the presence of a sufficient amount of FGF-2 on specific target cells that are mainly endothelial cells.

Angiogenesis has been shown to be dysregulated in several diseases, such as in coronary artery (CA) aneurysms in the chronic phase of Kawasaki disease (KD). Significant neovascularization occurs in acute KD CA aneurysms and myocardium soon after onset of the disease and multiple angiogenesis factors are involved, and that dysregulation of angiogenesis likely contributes to KD vasculopathy (Freeman et al, 2005, Pediatr Cardiol). Counteracting FGF2 activity may represent a promising therapeutic target for this disease.

According to a third embodiment, the peptide according to the invention is used as a drug in cancer therapy, in particular in solid tumors therapy.

Cancer generally refers to one of a group of diseases caused by the uncontrolled, abnormal growth of cells that can spread to adjoining tissues or other parts of the body. In particular, cancer cells present uncontrolled proliferation, loss of specialized functions, immortality, metastatic potential, rapid growth and proliferation rates, and specific morphological features and cellular markers. Cancer cells can form a solid tumor, in which the cancer cells are massed together in a specific site of the body.

In a specific aspect, the peptide used as a drug in cancer therapy is intended to treat one of the most common cancers, including breast cancer, lung cancer, prostate cancer, colorectal cancer, stomach cancer, skin cancer, brain cancer and cervical cancer.

Features of the Peptide

The present invention is related to a peptide comprising an amino acid sequence at least 85% identical to the amino acid sequence as shown in SEQ ID NO. 1, wherein the residues Lys⁹⁰⁵, Arg⁹⁰⁹, and Arg⁹¹² contained therein are present, for use as a medicament.

In a specific aspect of the invention, the peptide comprises a sequence as shown in SEQ ID NO. 2 [X-K⁹⁰⁵-X-X-X-X-X-R⁹⁰⁹-X-X-R-X-X-X-X-X-X-X-X-X-X-X], wherein X represents any amino acid. This sequence of twenty amino acids comprises the conserved residues Lys⁹⁰⁵, Arg⁹⁰⁹, and Arg⁹¹² that are important for the activity of the peptide as a medicament.

The reference sequence SEQ ID NO. 1 consists in 127 residues. Among these residues, a specific site of binding to heparin has been identified where the contribution of the conserved residues Lys⁹⁰⁵, Arg⁹⁰⁹, Arg⁹¹² is essential (see the examples section); beside, the residues Arg⁹¹⁸ and Arg⁹²¹ have been identified, even if not absolutely necessary, as playing a role in the binding activity to heparin (Ricard-Blum et al., 2006).

The peptide according to the invention comprises these essential amino acids, and otherwise can be modified, in particular by deletion, addition or substitution of residues, in the limits of 85% of identity with the reference sequence as shown in SEQ ID NO. 1. In particular, the peptide can be modified in order to increase its half-life, to increase its bioavailability and/or to make it less susceptible to proteolysis. These modifications may include cyclization of the peptide, incorporation of D-amino acids, or incorporation of non-natural amino acids. None of the modifications should substantially interfere with the desired biological activity of the peptide.

In a specific aspect of the invention, the peptide comprises the amino acid sequence as shown in SEQ ID NO. 3:

G-K-P-G-P-R-G-Q-R-G-P-T-G-P-R-G-E-R-G-P

According to this embodiment, the peptide comprises a sequence that presents 100% of identity with the sequence of twenty amino acids of SEQ ID NO. 3, comprised between the residue G⁹⁰⁴ and the residue P⁹²³, and other residues in the N-terminal and C-terminal portions.

In a preferred embodiment of the invention, the peptide has an amino acid sequence that consists in the sequence as shown in SEQ ID NO. 1.

The peptide can be prepared by all means known by the man skilled in the art, for example by chemical synthesis, or by using living systems such bacteria, yeast or eukaryote cells, such as animal and plant cells. Preferred microorganisms for the synthesis of the peptide are E. coli and yeasts.

Accordingly, a vector carrying a molecule of nucleic acid encoding the peptide is introduced to a bacteria or eukaryote cell, by any suitable technique of transformation. Microorganisms are then grown under constant agitation in a suitable medium, in a suitable temperature, for example 37° C., and produce the peptide such as encoded by the vector. Said peptide is then purified, for example on ion exchange columns, before being used as a medicament. In particular, the purified peptide is analyzed by mass spectrometry to check that no bacterial contaminants are present in the purified sample.

According to the invention, the term ‘peptide’ always designates a ‘purified’ or ‘isolated’ peptide, which indicates that the peptide has been separated from other components such as proteins and organic molecules that are naturally present in a growth medium for bacteria.

In a specific embodiment of the invention, the peptide is coupled to a detectable label.

A detectable label designates a compound that is “detectable” in particular in an imaging procedure, because it is colored, fluorescent or luminescent. In a particular embodiment, the detectable label is chosen among a radioactive label, an affinity label, a magnetic particle, a fluorescent or luminescent label.

In particular, the detectable moiety may be a contrast agent or a detectable protein. The man skilled in the art knows several detectable proteins such as the Green Fluorescent Protein, and several fluorescent dyes such as the Alexa Fluor family.

The detectable label may be a fluorescent protein. In particular, if the peptide is produced in a living system, the vector carrying nucleic acid encoding the peptide includes also nucleic acid encoding such fluorescent protein.

In a specific aspect of the invention, both nucleic acids are organized on the vector under the control of the same promoter, to be transcribed and translated together, in a way to form a fusion protein comprising both the peptide and the detectable protein.

In another aspect of the invention, the peptide is chemically fused to a chromophore group.

Advantageously, the peptide can be followed in a body of animal or patient, by in vivo imaging, by techniques well known by the man skilled in the art.

Administration of the Peptide

In a preferred aspect of the invention, in its use as a medicament, an effective amount of the peptide is administered to an animal or an individual, and an accumulation of said peptide in the angiogenesis or tumor site(s) is obtained.

The “effective amount” of the peptide refers to the amount necessary to elicit the desired biological response. As can be appreciated by the man skilled in the art, the effective amount may vary depending on factors such as the desired biological endpoint, the structure of the peptide, and/or the target tissue.

The peptide can be administered by any route of administration. Suitable routes may include oral, buccal, by inhalation spray, sublingual, rectal, transdermal, vaginal, transmucosal, nasal or intestinal administration, parenteral delivery, including intramuscular, subcutaneous and intravenous injections, or other modes of delivery.

A preferred mode of administration is the parental administration into the blood system of the animal or individual.

In the case of cancer treatment, the angiogenesis site(s) are mainly the sites surrounding the solid tumors, where the dynamic angiogenesis process is stimulated, in particular by the presence of FGF-2.

The present invention relates in particular to a peptide as described above, for its use for treatment of cancer, by administration of an effective amount of said peptide to an animal or an individual, whereby an accumulation of said peptide in the angiogenesis or tumor site(s) is obtained.

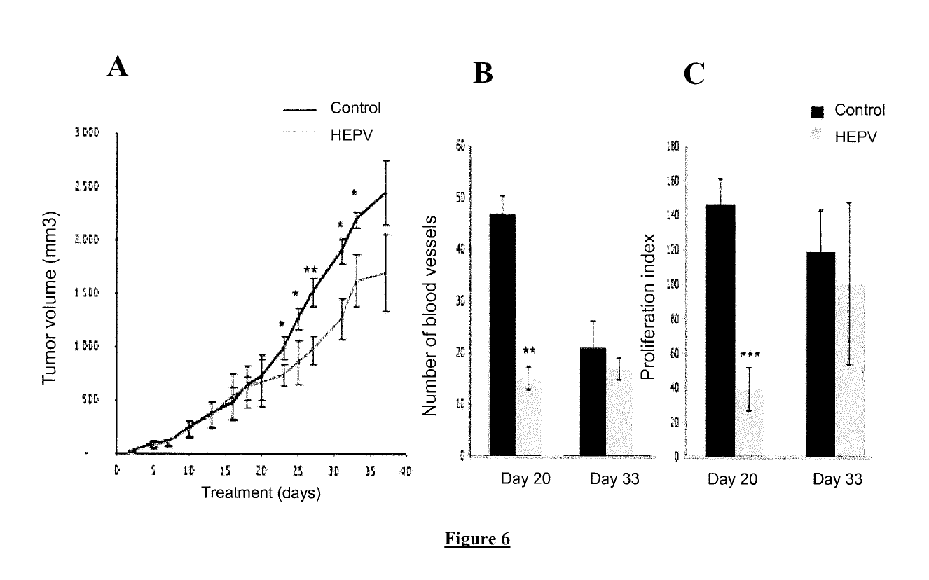

Example 4 and FIG. 5A below demonstrate that, when injected into the blood system of a mouse, the peptide HEPV comprising the amino acids Lys⁹⁰⁵, Arg⁹⁰⁹, and Arg⁹¹² accumulates in the site of angiogenesis, although the control peptide where the three essential amino acids have been replaced with alanine does not.

Pharmaceutical Compositions and Kits-of-Part

The present invention also relates to a pharmaceutical composition comprising an effective amount of at least one peptide as described above, and a pharmaceutically acceptable vehicle.

A pharmaceutically acceptable vehicle is a physiologically acceptable vehicle prepared with nontoxic components, useful for administering an active compound to an animal or a patient in need.

The pharmaceutical composition may comprise different peptides, in particular at least two types of peptides selected from the presently disclosed peptides.

This pharmaceutical composition may further comprise a compound inhibiting angiogenesis, in particular a compound inhibiting VEGF-induced angiogenesis.

This pharmaceutical composition comprising an effective amount of at least one peptide as described above, and a pharmaceutically acceptable vehicle may further comprise an anti-inflammatory compound.

This pharmaceutical composition comprising an effective amount of at least one peptide as described above, and a pharmaceutically acceptable vehicle may further comprise an anticancer active ingredient.

This pharmaceutical composition comprising an effective amount of at least one peptide as described above, and a pharmaceutically acceptable vehicle may further comprise a compound inhibiting VEGF-induced angiogenesis and an anti-inflammatory agent.

This pharmaceutical composition comprising an effective amount of at least one peptide as described above, and a pharmaceutically acceptable vehicle may further comprise a compound inhibiting angiogenesis and an anticancer active ingredient.

This pharmaceutical composition comprising a an effective amount of at least one peptide as described above, and a pharmaceutically acceptable vehicle may further comprise a compound inhibiting angiogenesis, an anti-inflammatory agent and an anticancer active ingredient.

Advantageously, after administration of the pharmaceutical composition in the blood system of an animal or an individual, an accumulation of said peptide in the angiogenesis or tumor site(s) is obtained. This feature, as shown in the FIG. 5A, is highly advantageous for the use of said peptide as a medicament.

The present invention also relates to a kit-of-parts comprising an effective amount of the peptide as described above, and another compound inhibiting angiogenesis, in particular a compound inhibiting VEGF-induced angiogenesis, and/or an anti-inflammatory compound, and/or an anticancer active ingredient.

Said kit-of-parts allows the administration to a patient of the peptide and a compound inhibiting angiogenesis, and/or an anti-inflammatory compound, and/or an anticancer active ingredient, at different times. The administration of these two or three components can be realized concomitantly or sequentially.

In particular, the kit-of-parts comprises an effective amount of the peptide as described above, and a compound inhibiting angiogenesis.

In another embodiment, the kit-of-parts comprises an effective amount of the peptide as described above, and an anticancer active ingredient, such as a chemotherapy compound.

In another embodiment, the kit-of-parts comprises an effective amount of the peptide as described above, and an anti-inflammatory compound.

In another embodiment, the kit-of-parts comprises an effective amount of the peptide as described above, a compound inhibiting angiogenesis, and an anticancer active ingredient, such as a chemotherapy compound.

In another embodiment, the kit-of-parts comprises an effective amount of the peptide as described above, a compound inhibiting angiogenesis and an anti-inflammatory compound.

In another embodiment, the kit-of-parts comprises an effective amount of the peptide as described above, a compound inhibiting angiogenesis, an anti-inflammatory compound, and an anticancer active ingredient. In particular, the patient may be treated in a first step with a compound inhibiting angiogenesis, and/or an anti-inflammatory compound, and/or an anticancer active ingredient; if it appears that the patient still presents an active angiogenesis process around the tumors, in a second step of the treatment, the peptide according to the invention is administered, with or without an anticancer active ingredient.

Advantageously, the patient may be further treated with other methods. Such methods may include, but are not limited to, chemotherapy, radiation therapy or surgery.

The administration of a pharmaceutical composition of the present invention may be conducted before, during or after other cancer therapies.

The present invention also relates to a method for inhibiting the biological effects of FGF-2 on target cells in vitro or ex vivo, comprising contacting the cells with an effective amount of a peptide comprising an amino acid sequence at least 85% identical to the amino acid sequence as shown in SEQ ID NO. 1 [Ile⁸²⁴-Pro⁹⁵⁰], wherein the residues Lys⁹⁰⁵, Arg⁹⁰⁹ and Arg⁹¹² contained therein are present.

In another aspect of the invention, in this peptide, five residues Lys⁹⁰⁵, Arg⁹⁰⁹ Arg⁹¹², Arg⁹¹⁸ and Arg⁹²¹ are conserved.

The method as described above is performed, in particular, wherein the peptide comprises a conserved sequence as shown in SEQ ID NO. 2 [X-K⁹05-X-X-X-R⁹⁰⁹-X-X-R⁹¹²-X-X-X-X-X-X-X-X-X-X-X], wherein X is any amino acid.

In a specific aspect of the invention, the peptide comprises the conserved amino acid sequence as shown in SEQ ID NO. 3 [G-K⁹⁰⁵-P-G-P-R⁹⁰⁹-G-Q-R⁹¹²-G-P-T-G-P-R⁹¹⁸-G-E-R⁹²¹-G-P].

In a preferred embodiment of the invention, the peptide used in this method has an amino acid sequence that consists in the sequence as shown in SEQ ID NO. 1.

Labeled Peptide and its Uses

The present invention also relates to a peptide comprising an amino acid sequence at least 85% identical to the amino acid sequence as shown in SEQ ID NO. 1, wherein the residues Lys⁹⁰⁵, Arg⁹⁰⁹, and Arg⁹¹² contained therein are present, coupled to a detectable label.

In another aspect of the invention, in this labelled peptide, five residues Lys⁹⁰⁵, Arg⁹⁰⁹ Arg⁹¹², Arg⁹¹⁸ and Arg⁹²¹ are conserved.

In a specific aspect of the invention, the labelled peptide comprises a conserved sequence as shown in SEQ ID NO. 2 [X-K⁹⁰⁵-X-X-X-R⁹⁰⁹-X-X-R⁹¹²-X-X-X-X-X-X-X-X-X-X-X], wherein X is any amino acid.

In a specific aspect of the invention, the labelled peptide comprises the conserved amino acid sequence as shown in SEQ ID NO. 3 [G-K⁹⁰⁵-P-G-P-R⁹⁰⁹-G-Q-R⁹¹²-G-P-T-G-P-R⁹¹⁸-G-E-R⁹²¹-G-P].

In a preferred embodiment of the invention, the labelled peptide has an amino acid sequence that consists in the sequence as shown in SEQ ID NO. 1.

The detectable label is in particular a radioactive label, an affinity label, a magnetic particle, a fluorescent or luminescent label. The detectable label may be a fluorescent protein.

In another aspect of the invention, the peptide is chemically fused to a chromophore group.

Advantageously, the peptide can be followed in a body of animal or patient, by in vivo imaging, by techniques well known by the man skilled in the art.

The present invention also relates to the use of the labelled peptide as described above, as an imaging agent, in particular to be used in vivo.

The present invention also relates to a method for imaging angiogenesis sites of an animal or of a human individual, comprising the step of detecting the label of a peptide as defined previously, that have been previously administered to the said animal or to the said human individual.

The present invention also relates to a method for imaging angiogenesis sites of an animal, comprising the steps of

(a) coupling a detectable label with at least one peptide such as described above,

(b) administering said labelled peptide to said animal, and

(c) detecting the label after sacrifice of the animal.

In a preferred aspect of the invention, in its use as an imaging agent in vivo, an effective amount of the peptide is administered to an animal or a human individual, and an accumulation of said peptide in the angiogenesis or tumor site(s) is obtained.

The peptide can be administered by any route of administration. Suitable routes may include oral, buccal, by inhalation spray, sublingual, rectal, transdermal, vaginal, transmucosal, nasal or intestinal administration, parenteral delivery, including intramuscular, subcutaneous and intravenous injections, or other modes of delivery.

The detection of the label is performed by any technique well known by the man skilled in the art.

### EXAMPLES

Material and Methods

Preparation and Expression and Purification of HEPV and ΔHBS-HEPV

The recombinant HEPV fragment and ΔHBS-HEPV construct were prepared as previously described and inserted into the EcoRI and PstII sites of the pT7/7 expression vector. The pR905A plasmid previously obtained, where the arginine at position 905 was replaced by an alanine, was used as a template to generate the triple mutant R905/R909/R912 by using the QuikChange II site-directed mutagenesis kit (Stratagene, UK). Point mutations were introduced with the oligonucleotides:

(SEQ ID NO. 6)
5′-GCGCCCAGGACCGGCGGGGGCAGGCAGGCCCAACG-3′,
and
(SEQ ID NO. 7)
5′-CGTTGGGCCTGCCTGCCCCGCCGGTCCTGGCGC-3′.

The identity of ΔHBS-HEPV was verified by nucleotide sequencing. The recombinant wild-type plasmid named pHEPV and the mutant pAHBS-HEPV obtained were transformed in an E. coli strain (BL21 SI-GJ1158) that carries the T7 RNA polymerase gene under the control of the salt inducible proU promoter. After a 20 h induction with 0.2 M NaCl, cells were harvested by centrifugation and resuspended in 50 mM Tris-HCl, pH 7.4, and then sonicated. After centrifugation and filtration, bacterial supernatants were first subjected to cation exchange chromatography using a HiTrapSP column (Amersham) to remove most contaminant bacterial proteins and were purified to homogeneity using a Mono Q column (Amersham). Recombinant protein containing fractions were analyzed by SDS-PAGE on a 15% gel and dialyzed against 50 mM Tris-HCl, pH 7.5. The recombinant HEPV fragment and ΔHBS-HEPV were stored at −20° C. until use.

Heparin Affinity Chromatography

Heparin-Sepharose affinity columns (HiTrap Heparin, Amersham) were equilibrated in 50 mM Tris-HCl (pH 7.4). Protein samples were loaded onto a column, and a programmed linear gradient of 0-500 mM NaCl, 1M Tris-HCl (pH 7.4) was applied at a flow rate of 0.5 ml/min, confirmed by continuous conductivity measurement. Fractions (1 ml) were collected, and the elution profile of protein samples was determined by monitoring the absorbance at 214 nm. To accurately compare elution positions of the mutant, its elution with NaCl gradient was achieved versus a standard HEPV elution.

Quantitative Real-Time RT-PCR

Total RNA was isolated from 8×10⁶ cells by phenol-chloroform-isopropanol extraction (Trizol Reagent, Invitrogen). A reverse transcriptase reaction was performed on 1 μg of RNA using M-MLV reverse transcriptase (Promega). Quantitative PCRs were performed using SYBR Green Supermix (Biorad) and specific primers using a I-Cycler Optical System (Biorad). The following primers were used:

COL4A1 forward
(SEQ ID NO. 8)
5′-CTGGTCCAAGAGGATTTCCA-3′;
COL4A1 reverse
(SEQ ID NO. 9)
5′-TCATTGCCTTGCACGTAGAG-3′;
COL18A1 forward
(SEQ ID NO. 10)
5′-GCGCCAAAGGAGAAGTGG-3′;
COL18A1 reverse
(SEQ ID NO. 11)
5′-TTTCAGCCTCCAACTGAAGAA-3′;
L30 forward
(SEQ ID NO. 12)
5′-ATGGGGAAGGTGAAGGTCG-3′;
and
L30 reverse
(SEQ ID NO. 13)
5′-TAAAAGCAGCCCTGGTGACC-3′.

L30 was selected as housekeeping gene and used for normalization. Relative transcript abundances were determined as well known by the man skilled in the art. Relative gene expression was determined using the 2^{-ΔΔC}^{_{T}} method. Student's t-tests were used to determine statistical significance (n=4).

Phosphorylation Assays

HUVEC were seeded at 2.10⁵ cells/wells in ECGM2 in 6-well plates. The cells were treated with HEPV or ΔHBS-HEPV (8 μg/mL) during 24 h in serum free medium and then stimulated with FGF-2 or VEGF (50 ng/mL) for 5 or 20 minutes at 37° C. Cells were then washed twice with cold PBS and scraped and lysed at 4° C. in lysis buffer 1% NP-40 (150 mM NaCl, 50 mM Hepes pH 7.4, 5 mM EDTA, 10% glycerol, 1% NP-40, complete protease inhibitor cocktail (Roche), 1 mM Na₃VO₄). After centrifugation (13 000 g, 15 min, 4° C.), soluble proteins were collected. Protein amount was determined by BCA protein assay (Pierce) and equal amounts of proteins (10 μg) were loaded on SDS-PAGE and transfer on PVDF membrane at 100 V during 1 h. The blots were blocked with 5% BSA in TBS-T buffer (20 αmM Tris-HCl pH 7.4 and 0.05% Tween 20) and incubated with primary antibodies in TBST containing 5% bovine serum albumin, overnight at 4° C. Immunoreactivity was detected by sequential incubation with horseradish peroxidase-conjugated secondary antibodies purchased and ECL detection reagents purchased from Biorad. The antibodies used for phospho-protein detection were the followings: anti-AKT, anti-phospho-Akt (Ser473), anti-ERK1/2 and anti-phospho-ERK1/2 (Thr202/Tyr204, Thr185/Tyr187) (all from Cell Signaling Technology).

Animal Experiments

All animal experiments were performed in agreement with the EEC guidelines and the Principles of laboratory animal care (NIH publication 14, no. 86-23, revised 1985); the protocol was approved by the Animal Care and Use Committee. Female athymic NMRI nude mice (Janvier, Le Genest-Isle, France) were used in this study and maintained under specific pathogen-free conditions. Local surgery was performed under general anesthesia, which was induced via intraperitoneal injection of Domitor (Pfizer, Orsay, France) and Imalgene® (Merial, Lyon, France).

Implantation of the Sponges

Disc Cellspon cellulose sponges (thickness 2 mm, diameter 10 mm; Cellomeda, Turku, Finland) were implanted under the skin of the mice. Prior to implantation, the sponges were hydrated with 50 μl of either PBS (negative control) or FGF-2 (200 ng/50 μl; positive angiogenic control) (recombinant FGF-2, Eurobio-AbCys, Les Ulis, France). After implantation, the sponges were re-injected through the skin on days 1 and 2 with 50 μl PBS either without (negative control) or with 200 ng FGF-2 (positive control) in the absence and presence of the HEPV peptides to be tested.

2D Fluorescence In Vivo Imaging

Angiogenesis was examined on day 7. For 2D fluorescence imaging, 200 μl HEPV-Cy5 or HEPVAHBS-AlexFluo700 were injected intravenously (50 fig) into the mouse tail vein and imaged 3 h post-injection. Mice were illuminated with 660-nm light-emitting diodes equipped with interference filters and fluorescence images, as well as black and white pictures, which were acquired by a back-thinned charge-coupled device (CCD) camera at −80° C. (ORCAII-BT-512G; Hamamatsu, Massy, France), fitted with a high-pass RG 9 filter (Schott, Clichy, France). An ROI was then positioned on the sponge in order to measure the number of photons/pixel during 200 ms.

Hemoglobin Measurements

After implantation of the PBS or FGF-2 treated sponges and treatment with 50 μl containing 50 μg HEP peptides at DO, 2, 3 and 5, the hemoglobin content was measured at D8. Mice were sacrificed via a lethal injection of Doletal®, and the sponges were then rapidly excised and photographed. Each sponge was homogenized in 1 ml of RIPA lysis buffer containing a cocktail of protease inhibitors, centrifuged at 200 g, and the supernatants were quantified. The extent of vascularization of the sponge implants was assessed by measuring the concentration of hemoglobin with Drabkin's reagent (Sigma-Aldrich, Saint-Quentin Fallavier, France). The results are expressed in mg/ml.

Antitumor Activity

TS/Apc-pGL3 is a cell line derived from the original adenocarcinoma TS/Apc mouse cell line stably transfected with the pGL3-luciferase reporter gene (Promega, Charbonnieres, France). Cells were cultured at 37° C. in a humidified 5% CO₂ incubator in RPMI 1640 supplemented with 1% glutamine, 10% fetal bovine serum, 50 units/ml penicillin, 50 μg/ml streptomycin, β-mercaptoethanol (25 μM) and 700 μg/ml Geneticin® (G418 sulphate; Gibco, Paisley, UK).

Twenty female athymic Swiss nude mice, purchased from Janvier (Le Genest Saint Isle, France) at 6-8 weeks of age were used and maintained under specific pathogen-free conditions. Mice received a subcutaneous (s.c.) injection of 10⁶ TS/Apc-pGL3 cells suspended in 200 μl of PBS into the right flank usually results in formation of 6-8 mm-diameter tumors after one week. Starting at day 5 after sc implantation, mice received 2 peritumoral injection every 2 days of 50 μl pf PBS solution containing 50 μg of HEP (n=10) or HEPVAHBS (n=10) peptides. Tumor growth was evaluated using calipers.

At day 20, 3 mice/group were sacrificed for immunohistology studies. At day 35 days after implantation all remaining tumors were extracted.

Frozen sections (8 μm) from tumors were fixed in acetone for 10 min. Sections were then washed 3×5 min in Tris-buffered saline containing 0.1% Tween 20, and endogenous peroxidases were blocked with 0.1% H₂O₂ in methanol for 20 min. Sections were then sequentially incubated for 1 h with a rat monoclonal anti-CD31 antibody (MEC13.3;1:500; Pharmingen) or rabbit anti-Ki67 (1:100; AbCAM) and for 1 h with goat anti-rat antibody for CD31 (1:500; Cell-signaling) or goat anti-rabbit for Ki67 (1:200; Dako). Peroxidase activity was revealed using diaminobenzidinetetrachloride as a chromogen (Dako; San Antonio, Tex., USA). Sections were counterstained with hematoxylin and all were mounted.

Staining against the endothelial marker CD31 by means of immunohistochemistry was followed by observation under low magnification scope (100×), five field of view of each tumor (5 tumors by condition). Then, vessels quantity were measured in each of these areas utilizing ImageJ software (http://rsbweb.nih.gov/ij). All counts were performed in a blinded manner.

After immunohistochemical staining against Ki67, slides were observed under high magnification scope (200×). Six to nine areas by tumor were photographed. These photographs were analyzed by the plugging ImmunoRatio in ImageJ software (http://rsbweb.nih.gov/ij). The Ki67 index was evaluated in a blinded manner and calculated as Ki67-positive cells divided by all tumor cells in one field.

### Example 1. Role of HEPV on the Expression of Collagens

A transcriptomic analysis has been performed, in order to identify the HEPV-regulated genes. Endothelial HDMEC cells have been treated with HEPV or not, for 4, 12 and 24 hours. In the list of 219 up-regulated genes, the genes COL4A1 and COL18A1, coding for the α1 chains of collagens IV and XVIII respectively, have been identified as being very relevant, since they encode proteins located in the vascular endothelial basal membranes and that possess a strong anti-angiogenic activity after cleavage.

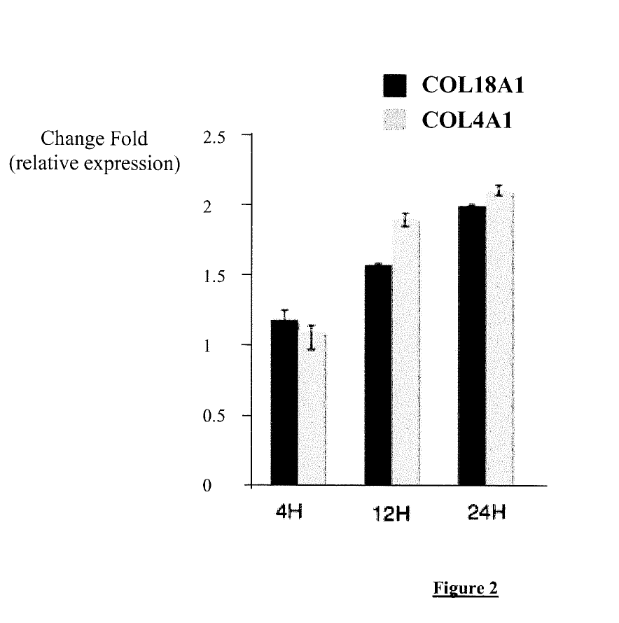

Up-degulation of these genes has been validated by quantitative PCR (FIG. 2).

It appears therefore that HEPV induces the expression of proteins involved in the control of the angiogenesis process.

### Example 2. Preparation of a Non-Functional Mutant of HEPV: ΔHBS-HEPV

The peptide HEPV presents the sequence as shown in SEQ ID NO. 1.

The peptide ΔHBS-HEPV presents the sequence as shown in SEQ ID NO. 4, wherein the following residues has been replaced with alanines: Lys⁹⁰⁵, Arg⁹⁰⁹ and Arg⁹¹².

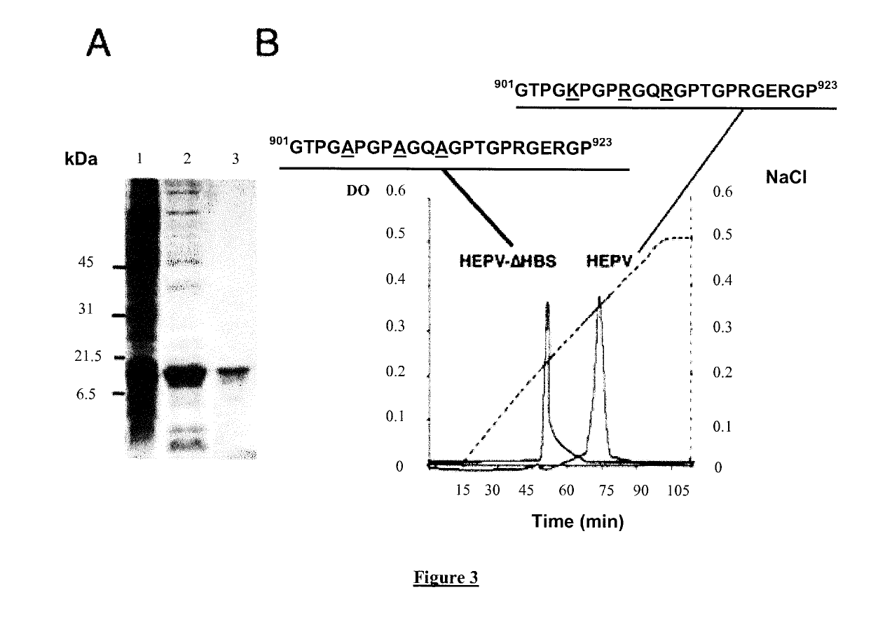

Both peptides have been produced in a bacterial system of Escherichia coli. The peptide ΔHBS-HEPV is purified on ion-exchange chromatographic columns. FIG. 3A show the supernatant of Escherichia coli growth medium before (line 1), after a first step (line 2) and a second step of column purification (line 3).

The affinity of both peptides for heparin is compared, on a heparin-sepharose column, determined with the necessary quantity of NaCl to elute the peptides.

Although the HEPC peptide is eluted with a concentration of 0.35M NaCl, the mutated peptide ΔHBS-HEPV is eluted with a concentration of 0.2M (FIG. 3B), close to the physiologic concentration of NaCl (0.15M).

It appears therefore that the mutant presents a significant disability to bind to heparin, compared to the peptide HEPV, and is suitable to be used in the next experiments as a negative control.

### Example 3. HEPV Acts on Signalization Pathway of FGF-2 and VEGF

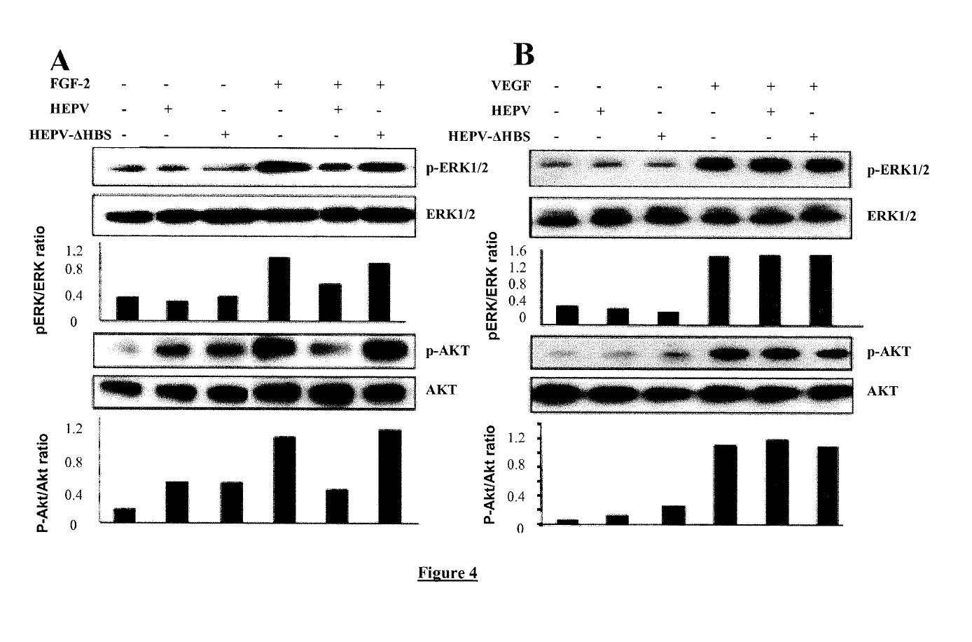

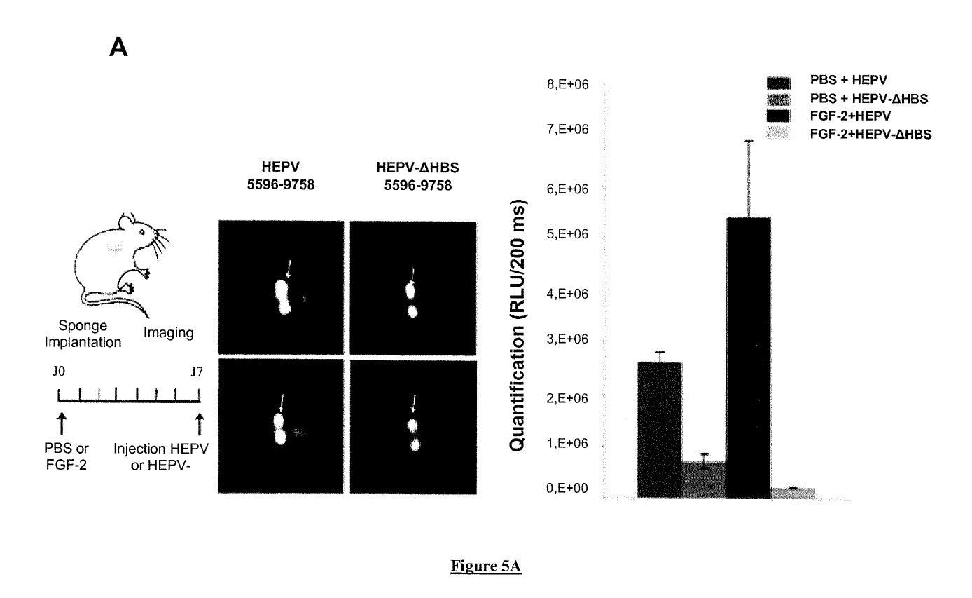

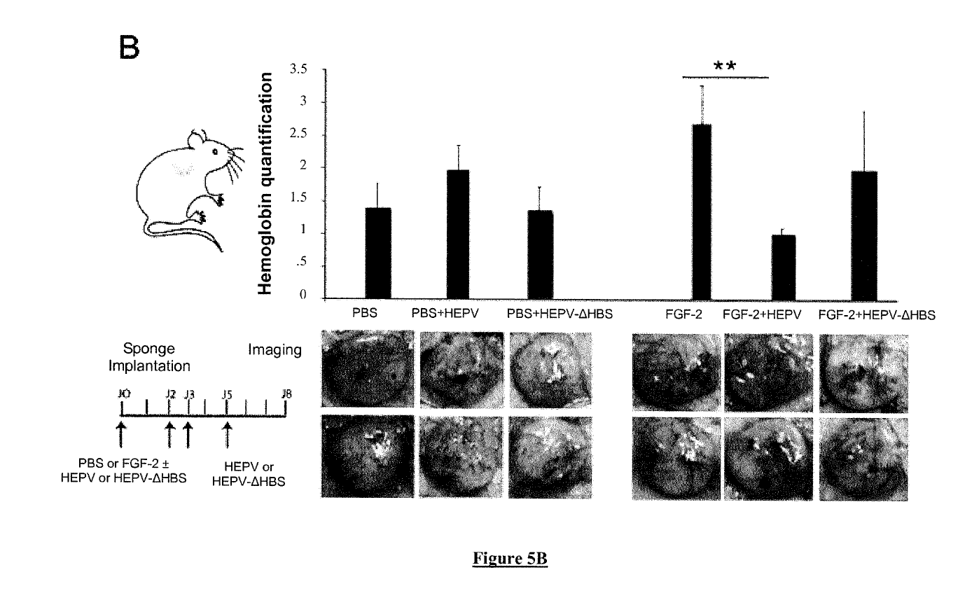

The aim of the experiments presented in FIG. 4 was to determine if, after incubation of endothelial cells with the peptide HEPV, the response to FGF-2 was affected by the presence of this peptide. The measured response to FGF-2 is the level of phosphorylation of proteins ERK 1/2 and Akt, involved in the signalization pathway of FGF-2 and VEGF, as determined with specific antibodies.

Endothelial cells HUVEC have been treated during 24 hours with HEPV or the control peptide ΔHBS-HEPV, and have then been stimulated with FGF-2 (FIG. 4A) or VEGF (50 ng/ml) (FIG. 4B).

Non-treated cells present a significant increase of the phosphorylation of ERK1 (line p-ERK1/2 for ‘phosphorylated ERK1/2’) after stimulation with FGF-2. This phosphorylation is inhibited in cells treated with HEPV. On the contrary, the control peptide ΔHBS-HEPV is inefficient for inhibiting the phosphorylation of ERK1/2 (FIG. 4A).

This action of HEPV is FGF-2-specific since all cells stimulated with VEGF present a phosphorylation of ERK1/2, even after treatment with HEPV (FIG. 4B).

Similar results are observed for the phosphorylation level of Akt protein, after stimulation with FGF-2 and VEGF.

### Example 4. HEPV Acts on Formation of Blood Vessels In Vivo in Mouse

In nude mice, a cellulose sponge has been implanted under the skin, this sponge containing an angiogenesis factor (FGF-2), in order to artificially stimulate angiogenesis. Negative control sponges comprise PBS.

HEPV and ΔHBS-HEPV peptides have been fused to a fluorophore (Alexa Fluor 700) and have been injected to the blood system in mice. To follow the localization of the fusion proteins in vivo, pictures have been realized every 3 hours.

The quantification of the fluorescence in the site of the sponge (FIG. 5A) indicates that:
for sponges impregnated with FGF-2, stimulating angiogenesis, the fusion protein HEPV-fluo accumulates strongly in the sponge;
on the contrary, the control fusion protein ΔHBS-HEPV-fluo does not accumulate in the sponge.

Moreover, in sponges impregnated with FGF-2, the number of newly formed blood vessels is twice the number observed in control sponges (FIG. 5A, on the right).

Other results, not shown, demonstrate that the peptide HEPV does not accumulate non-specifically in various organs, except in kidney and bladder, the elimination specialized-organs. Moreover the peptide does not accumulate into the liver, and is therefore non-toxic.

FIG. 5B shows the results in terms of anti-angiogenesis activity of HEPV. 20 μg of HEPV or ΔHBS-HEPV have been injected simultaneously with PBS or FGF-2, at the first day and then every two days. After 7 days of treatment, sponges are taken out and analyzed. The level of angiogenesis is measured via the level of hemoglobin found in sponges. When mice have been treated with HEPV, the hemoglobin level is significantly decreased (of 2.5 times) in comparison with mice treated with ΔHBS-HEPV (FIG. 5B, on the right).

These results show that (i) HEPV peptide is able to inhibit FGF-2 induced angiogenesis; and that (ii) a functional binding site to heparin is necessary for obtaining this effect.

### Example 5. HEPV Affects the Tumoral Growth

Tumors have been induced by implantation of murine breast cancer cells (TSA) in nude mice. Once the tumors are developed, an intra-tumoral injection of HEPV (50 μg) is realized every two days, during 37 days. The volume of the tumors is measured with a caliper.

Results are shown in FIG. 6A. Up to day 18, all tumors develop according to the same model. From day 20 of treatment, the growth of the tumors treated with HEPV slows down, up to the end of the experiment.

Mice from each group are sacrificed at day 20 and day 33 to check the formation of novel blood vessels. The account of the blood vessels is realized by observations of the tumors samples.

Results are shown in FIG. 6B. At day 20, a significant decrease of the blood vessels density is observed in mice treated with HEPV, when compared to ΔHBS-HEPV-treated-mice. Curiously, at day 33, the difference is less significant. A possible explanation is the fact that at this step of the treatment, the presence of necrosis zones does not allow a right follow-up of the angiogenesis process.

The proliferation of tumor cells with an antibody anti-Ki67 is also realized on these tumor samples.

Results are shown in FIG. 6C. At day 20, a significant decrease of the tumor cells proliferation is observed. However, at day 33, it appears that the proliferation strikes back.

TABLE 1
Sequences

| SEQ ID | | |
|---|---|---|
| NO. | Name | SEQUENCE |
| 1 | HEPV (binding | IKGDRGEIGPPGPRGEDGPEGPKGRGGPNGDPGPLGPP |
| | site is underlined) | GEKGKLGVPGLPGYPGRQGPKGSIGFPGFPGANGEKGG |
| | | RGTPGKPGPRGQRGPTGPRGERGPRGITGKPGPKGNSG |
| | | GDGPAGPPGERGP |
| 2 | Minimal binding | X-K-X-X-X-R-X-X-R-X-X-X-X-X-X-X-X-X-X-X |
| | site of HEPV | |
| 3 | Wild-type binding | G-K-P-G-P-R-G-Q-R-G-P-T-G-P-R-G-E-R-G-P |
| | site of HEPV | |
| 4 | HEPV αHBS | IKGDRGEIGPPGPRGEDGPEGPKGRGGPNGDPGPLGPP |
| | | GEKGKLGVPGLPGYPGRQGPKGSIGFPGFPGANGEKGG |
| | | RGTPGAPGPAGQAGPTGPRGERGPRGITGKPGPKGNSG |
| | | GDGPAGPPGERGP |
| 5 | proα1(V) chain of | MDVHTRWKARSALRPGAPLLPPLLLLLLWAPPPSRAAQPADLLKVL |
| | the Collagen V | DFHNLPDGITKTTGFCATRRSSKGPDVAYRVTKDAQLSAPTKQLYP |
| | (HEPV peptide is | ASAFPEDFSILTTVKAKKGSQAFLVSIYNEQGIQQIGLELGRSPVFLY |
| | underlined) | EDHTGKPGPEDYPLFRGINLSDGKWHRIALSVHKKNVTLILDCKKK |
| | 1838 aa | TTKFLDRSDHPMIDINGIIVFGTRILDEEVFEGDIQQLLFVSDHRAAY |
| | | DYCEHYSPDCDTAVPDTPQSQDPNPDEYYTEGDGEGETYYYEYPY |
| | | YEDPEDLGKEPTPSKKPVEAAKETTEVPEELTPTPTEAAPMPETSEG |
| | | AGKEEDVGIGDYDYVPSEDYYTPSPYDDLTYGEGEENPDQPTDPGA |
| | | GAEIPTSTADTSNSSNPAPPPGEGADDLEGEFTEETIRNLDENYYDPY |
| | | YDPTSSPSEIGPGMPANQDTIYEGIGGPRGEKGQKGEPAIIEPGMLIE |
| | | GPPGPEGPAGLPGPPGTMGPTGQVGDPGERGPPGRPGLPGADGLPG |
| | | PPGTMLMLPFRFGGGGDAGSKGPMVSAQESQAQAILQQARLALRG |
| | | PAGPMGLTGRPGPVGPPGSGGLKGEPGDVGPQGPRGVQGPPGPAG |
| | | KPGRRGRAGSDGARGMPGQTGPKGDRGFDGLAGLPGEKGHRGDP |
| | | GPSGPPGPPGDDGERGDDGEVGPRGLPGEPGPRGLLGPKGPPGPPGP |
| | | PGVTGMDGQPGPKGNVGPQGEPGPPGQQGNPGAQGLPGPQGAIGP |
| | | PGEKGPLGKPGLPGMPGADGPPGHPGKEGPPGEKGGQGPPGPQGPI |
| | | GYPGPRGVKGADGIRGLKGTKGEKGEDGFPGFKGDMGIKGDRGEI |
| | | GPPGPRGEDGPEGPKGRGGPNGDPGPLGPPGEKGKLGVPGLPGYPG |
| | | RQGPKGSIGFPGFPGANGEKGGRGTPGKPGPRGQRGPTGPRGERGP |
| | | RGITGKPGPKGNSGGDGPAGPPGERGPNGPQGPTGFPGPKGPPGPPG |
| | | KDGLPGHPGQRGETGFQGKTGPPGPPGVVGPQGPTGETGPMGERG |
| | | HPGPPGPPGEQGLPGLAGKEGTKGDPGPAGLPGKDGPPGLRGFPGD |
| | | RGLPGPVGALGLKGNEGPPGPPGPAGSPGERGPAGAAGPIGIPGRPG |
| | | PQGPPGPAGEKGAPGEKGPQGPAGRDGLQGPVGLPGPAGPVGPPGE |
| | | DGDKGEIGEPGQKGSKGDKGEQGPPGPTGPQGPIGQPGPSGADGEP |
| | | GPRGQQGLFGQKGDEGPRGFPGPPGPVGLQGLPGPPGEKGETGDVG |
| | | QMGPPGPPGPRGPSGAPGADGPQGPPGGIGNPGAVGEKGEPGEAGE |
| | | PGLPGEGGPPGPKGERGEKGESGPSGAAGPPGPKGPPGDDGPKGSP |
| | | GPVGFPGDPGPPGEPGPAGQDGPPGDKGDDGEPGQTGSPGPTGEPG |
| | | PSGPPGKRGPPGPAGPEGRQGEKGAKGEAGLEGPPGKTGPIGPQGA |
| | | PGKPGPDGLRGIPGPVGEQGLPGSPGPDGPPGPMGPPGLPGLKGDSG |
| | | PKGEKGHPGLIGLIGPPGEQGEKGDRGLPGPQGSSGPKGEQGITGPS |
| | | GPIGPPGPPGLPGPPGPKGAKGSSGPTGPKGEAGHPGPPGPPGPPGEV |
| | | IQPLPIQASRTRRNIDASQLLDDGNGENYVDYADGMEEIFGSLNSLK |
| | | LEIEQMKRPLGTQQNPARTCKDLQLCHPDFPDGEYWVDPNQGCSR |
| | | DSFKVYCNFTAGGSTCVFPDKKSEGARITSWPKENPGSWFSEFKRG |
| | | KLLSYVDAEGNPVGVVQMTFLRLLSASAHQNVTYHCYQSVAWQD |
| | | AATGSYDKALRFLGSNDEEMSYDNNPYIRALVDGCATKKGYQKTV |
| | | LEIDTPKVEQVPIVDIMFNDFGEASQKFGFEVGPACFMG |
| 6 | Oligonucleotide | 5′-GCGCCCAGGACCGGCGGGGGCAGGCAGGCCCAACG-3′ |
| 7 | Oligonucleotide | 5′-CGTTGGGCCTGCCTGCCCCGCCGGTCCTGGCGC-3′ |
| 8 | COL4A1 forward | 5′-CTGGTCCAAGAGGATTTCCA-3′ |
| 9 | COL4A1 reverse | 5′-TCATTGCCTTGCACGTAGAG-3′ |
| 10 | COL18A1 forward | 5′-GCGCCAAAGGAGAAGTGG-3′ |
| 11 | COL18A1 reverse | 5′-TTTCAGCCTCCAACTGAAGAA-3′ |
| 12 | L30 forward | 5′-ATGGGGAAGGTGAAGGTCG-3′ |
| 13 | L30 reverse | 5′-TAAAAGCAGCCCTGGTGACC-3′ |

### REFERENCES

### Patents

US 2014/0100164
US 2013/0316950

### Bibliographic References

Alessi P, Leali D, Camozzi M, Cantelmo A, Albini A, Presta M. Anti-FGF2 approaches as a strategy to compensate resistance to anti-VEGF therapy: long-pentraxin 3 as a novel antiangiogenic FGF2-antagonist. Eur Cytokine Netw. 2009 December; 20(4):225-34.
Delacoux F, Fichard A, Geourjon C, Garrone R, Ruggiero F. Molecular features of the collagen V heparin binding site. J Biol Chem. 1998 Jun. 12; 273(24):15069-76.
Delacoux F, Fichard A, Cogne S, Garrone R, Ruggiero F. Unraveling the amino acid sequence crucial for heparin binding to collagen V. J Biol Chem. 2000 Sep. 22; 275(38):29377-82.
Ricard-Blum S, Beraud M, Raynal N, Famdale R W, Ruggiero F. Structural requirements for heparin/heparan sulfate binding to type V collagen. J Biol Chem. 2006 Sep. 1; 281(35):25195-204.
Haley E M, Kim Y. The role of basic fibroblast growth factor in glioblastoma multiforme and glioblastoma stem cells and in their in vitro culture. Cancer Lett. 2014 Apr. 28; 346(1):1-5.
Itoh N, Ohta H. Pathophysiological roles of FGF signaling in the heart. Front Physiol. 2013 Sep. 6; 4:247.
Tatebayashi Y, Lee M H, Li L, Iqbal K, Grundke-Iqbal I. The dentate gyrus neurogenesis: a therapeutic target for Alzheimer's disease. Acta Neuropathol. 2003 March; 105(3):225-32.
Chen C H, Poucher S M, Lu J, Henry P D. Fibroblast growth factor 2: from laboratory evidence to clinical application. Curr Vase Pharmacol. 2004 January; 2(1):33-43.
Katta K, Boersema M, Adepu S, Rienstra H, Celie J W, Mencke R, Molema G, van Goor H, Berden J H, Navis G, Hillebrands J L, van den Born J. Renal heparan sulfate proteoglycans modulate fibroblast growth factor 2 signaling in experimental chronic transplant dysfunction. Am J Pathol. 2013 November; 183(5):1571-84.
Le Bousse-Kerdilès M C, Chevillard S, Charpentier A, Romquin N, Clay D, Smadja-Joffe F, Praloran V, Dupriez B, Demory J L, Jasmin C, Martyré M C. Differential expression of transforming growth factor-beta, basic fibroblast growth factor, and their receptors in CD34+ hematopoietic progenitor cells from patients with myelofibrosis and myeloid metaplasia. Blood. 1996 Dec. 15; 88(12):4534-46.
Le Bousse-Kerdilès M C, Martyré M C. Dual implication of fibrogenic cytokines in the pathogenesis of fibrosis and myeloproliferation in myeloid metaplasia with myelofibrosis. Ann Hematol. 1999 October; 78(10):437-44. Review.
Freeman AF1, Crawford S E, Cornwall M L, Garcia F L, Shulman S T, Rowley A H. Angiogenesis in fatal acute Kawasaki disease coronary artery and myocardium. Pediatr Cardiol. 2005 September-October; 26(5):578-84.

1. A method for inhibiting the biological effects of FGF-2 on target cells in vitro or ex vivo, comprising contacting the cells with an effective amount of a peptide comprising an amino acid sequence at least 85% identical to the amino acid sequence as shown in SEQ ID NO. 1, wherein the residues Lys⁹⁰⁵, Arg⁹⁰⁹, and Arg⁹¹² are present.

2-4. (canceled)

5. The method according to claim 1, wherein the peptide comprises an amino acid sequence as shown in SEQ ID NO. 2.

6. The method according to claim 1, wherein the peptide comprises an amino acid sequence as shown in SEQ ID NO. 3.

7. The method according to claim 1, wherein the amino acid sequence of the peptide is a sequence as shown in SEQ ID NO. 1.

8. The method according to claim 1, wherein the peptide is produced in a living system, such as a bacteria, a yeast or an eukaryote cell.

9. The method according to claim 1, wherein the peptide is coupled to a detectable label.

10. The method according to claim 1, wherein by administration of an effective amount of said peptide to an animal or an individual, an accumulation of said peptide in the angiogenesis or tumor site(s) is obtained.

11. A pharmaceutical composition comprising
an effective amount of at least one peptide comprising an amino acid sequence at least 85% identical to the amino acid sequence as shown in SEQ ID NO: 1, wherein residues Lys⁹⁰⁵, Arg⁹⁰⁹ and Arg⁹¹² are present in the at least one peptide, and
a pharmaceutically acceptable vehicle.

12. The pharmaceutical composition of claim 11, further comprising another compound inhibiting angiogenesis, and/or an anti-inflammatory compound, and/or an anticancer active ingredient.

13. A kit-of-parts comprising an effective amount of a peptide comprising an amino acid sequence at least 85% identical to the amino acid sequence as shown in SEQ ID NO: 1, wherein residues Lys⁹⁰⁵, Arg⁹⁰⁹ and Arg⁹¹² are present in the peptide and another compound inhibiting angiogenesis, and/or an anti-inflammatory compound, and/or an anticancer active ingredient.

14. A peptide comprising an amino acid sequence at least 85% identical to the amino acid sequence as shown in SEQ ID NO. 1, wherein residues Lys⁹⁰⁵, Arg⁹⁰⁹, and Arg⁹¹² are present in the peptide, and wherein the peptide is coupled to a detectable label.

15. The peptide of claim 11, wherein the detectable label is selected from the group consisting of a radioactive label, an affinity label, a magnetic particle, a fluorescent and a luminescent label.

16. (canceled)

17. A method for imaging angiogenesis sites of an animal or of a human individual, comprising the step of detecting a detectable label of a peptide comprising an amino acid sequence at least 85% identical to the amino acid sequence as shown in SEQ ID NO: 1, wherein residues Lys⁹⁰⁵, Arg⁹⁰⁹ and Arg⁹¹² are present in the peptide, and wherein the peptide is coupled to the detectable label, that and wherein the peptide has been previously administered to the animal or to the human individual.

18. A method for imaging angiogenesis sites of an animal, comprising the steps of
a) coupling a detectable label to at least one peptide comprising an amino acid sequence at least 85% identical to an amino acid sequence as shown in SEQ ID NO: 1, wherein residues Lys⁹⁰⁵, Arg⁹⁰⁹ and Arg⁹¹² are present in the peptide,
b) administering the labelled peptide to the animal, and
c) detecting the detectable label after sacrifice of the animal.
